(19) United States
(12) Patent Application Publication
(10) Pub. No.: US 2025/0201004 A1
(43) Pub. Date: Jun. 19, 2025

(54) METHOD AND SYSTEM FOR DETECTION OF SPERM USING VIRTUAL-STAINING

(71) Applicant: THE GOVERNING COUNCIL OF THE UNIVERSITY OF TORONTO, Toronto (CA)

(72) Inventors: Harrison EDWARDS, Toronto (CA); Mohamed ELSAYED, Toronto (CA); Aaron Ray WHEELER, Toronto (CA)

(21) Appl. No.: 18/979,932

(22) Filed: Dec. 13, 2024

Related U.S. Application Data
(60) Provisional application No. 63/610,740, filed on Dec. 15, 2023.

(51) Int. Cl. G06V 20/69; G06T 7/73; G06V 10/26; G06V 10/774; G06V 10/776; G16B 20/00; G16H 30/40

(52) U.S. Cl. CPC G06V 20/698; G06T 7/73; G06V 10/26; G06V 10/774; G06V 10/776; G16B 20/00; G16H 30/40; G06T 2207/10056; G06T 2207/20021; G06T 2207/20081; G06T 2207/30024

(57) ABSTRACT

There is provided a system and method for detection of sperm using virtual-staining. The method including: receiving an unstained inference image, the inference image including a microscopic image capturing sperm cells; generating a virtual-stained image of sperm from the inference image using a trained generator machine learning model, the generator machine learning model taking the inference image as input, the generator machine learning model trained using a set of training images including microscopic images of sperm cells and a set of ground-truth images showing staining that identifies the sperm cells in the training images, the generator machine learning model trained by propagating determined losses between generated virtual-stained images and corresponding ground-truth images; and outputting the generated virtual-stained image of sperm.

### TECHNICAL FIELD

The following relates generally to microscopic image inspection, and more specifically, to a computer-based method and system for detection of sperm using virtual-staining.

### BACKGROUND

Sexual assault is a particular type of violent crime that is generally on the rise. A significant majority of sexual assault cases go unreported because of a lack of confidence in the justice system. In some jurisdictions, victims are often asked to pay thousands of dollars to process their own sexual assault kits. There is generally a public perception of slow analysis of forensic evidence in sexual assault cases which results in a reluctance among complainants to come forward. Rapid analysis, potentially even at the point of care (for example, at sexual assault centers and hospitals or police stations) would give victims more confidence to report assaults.

Various approaches for sperm examination and quantification require substantial expertise in, at least, cell staining, knowledge of sperm morphology, and significant wet lab time. Such systems mean that a sample must be acquired at one location, transported to another, queued until throughput is available, and finally processed by technicians with significant expertise. Significant challenges occur in such systems due to, for example, logistical deadlock, temporal latency, and human-resource depletion.

### SUMMARY

In an aspect of the present invention, there is provided a computer-implemented method for detection of sperm, the method comprising: receiving an unstained inference image, the inference image comprising a microscopic image capturing sperm cells; generating a virtual-stained image of sperm from the inference image using a trained generator machine learning model, the generator machine learning model taking the inference image as input, the generator machine learning model trained using a set of training images comprising microscopic images of sperm cells and a set of ground-truth images showing staining that identifies the sperm cells in the training images, the generator machine learning model trained by propagating determined losses between generated virtual-stained images and corresponding ground-truth images; and outputting the generated virtual-stained image of sperm.

In a particular case of the method, the staining that identifies the sperm cells in the ground-truth images comprises staining of only the sperm cells.

In another case of the method, the staining that identifies the sperm cells in the ground-truth images comprises at least two different types of stains.

In yet another case of the method, the generator machine learning model is trained using a first set of training images comprising microscopic images of sperm cells collected at a first time-period post-coitus and a first set of ground-truth images showing staining that identifies the sperm cells in the first set of training images, and wherein the generator machine learning model is further trained using a second set of training images comprising microscopic images of sperm cells collected at a later time-period post-coitus and a second set of ground-truth images showing staining that identifies the sperm cells in the second set of training images.

In yet another case of the method, the method further comprising determining locations of sperm cells in the generated virtual-stained image of sperm by applying an intensity threshold across locations of the generated virtual-stained image of sperm.

In yet another case of the method, the method further comprising determining a quantity of sperm cells in the generated virtual-stained image by determining contours around the locations having an intensity greater than the threshold, wherein each continuous region can be counted as a single sperm cell.

In yet another case of the method, the method further comprising determining a time interval between sperm deposition and sample collection of the sperm cells captured in the microscopic image using a second machine learning model, the second machine learning model takes as input the unstained inference image, the generated virtual-stained image of sperm, and the quantity of sperm cells in the generated virtual-stained image, the second machine learning model trained using samples collected at known intervals post-coitus.

In yet another case of the method, the generator machine learning model generates two or more virtual-stained images of sperm, at least two of the virtual-stained images of sperm comprising different virtual stains.

In yet another case of the method, the method further comprising determining locations of sperm cells in the generated virtual-stained image of sperm by applying an intensity threshold across locations of the generated virtual-stained image of sperm, wherein the locations of sperm cells being where two or more of the stains are above the intensity threshold.

In yet another case of the method, the generated virtual-stained image of sperm comprises one or more of virtual HY-LITER fluorescent staining, virtual DAPI (4′,6-diamidino-2-phenylindole) fluorescent staining, virtual haematoxylin and eosin staining, and a virtual picroindigocarmine staining.

In yet another case of the method, the method further comprising performing pre-processing on the unstained inference image, the pre-processing comprising dividing the inference image into tiles and providing each of the tiles as input to the generator machine learning model.

In another aspect, there is provided a system for detection of sperm, the system comprising one or more processors in communication with a data storage memory, the data storage memory comprising instructions for the one or more processors to execute: an input module to receive an unstained inference image, the inference image comprising a microscopic image capturing sperm cells; a training module to train a generator machine learning model using a set of training images comprising microscopic images of sperm cells and a set of ground-truth images showing staining of the sperm cells in the training images, the generator machine learning model trained by propagating determined losses between generated virtual-stained images and corresponding ground-truth images; an inference module to generate a virtual-stained image of sperm from the inference image using the trained generator machine learning model, the generator machine learning model taking the inference image as input; and an output module to output the generated virtual-stained image of sperm.

In a particular case of the system, the staining that identifies the sperm cells in the ground-truth images comprises staining of only the sperm cells.

In another case of the system, the staining that identifies the sperm cells in the ground-truth images comprises at least two different types of stains.

In yet another case of the system, the generator machine learning model is trained using a first set of training images comprising microscopic images of sperm cells collected at a first time-period post-coitus and a first set of ground-truth images showing staining that identifies the sperm cells in the first set of training images, and wherein the generator machine learning model is further trained using a second set of training images comprising microscopic images of sperm cells collected at a later time-period post-coitus and a second set of ground-truth images showing staining that identifies the sperm cells in the second set of training images.

In yet another case of the system, the one or more processors to further execute comprising a post-processing module to determine locations of sperm cells in the generated virtual-stained image of sperm by applying an intensity threshold across locations of the generated virtual-stained image of sperm.

In yet another case of the system, wherein the post-processing module further determines a quantity of sperm cells in the generated virtual-stained image by determining contours around the locations having an intensity greater than the threshold, wherein each continuous region can be counted as a single sperm cell.

In yet another case of the system, the one or more processors to further execute comprising a post-processing module to determine a time interval between sperm deposition and sample collection of the sperm cells captured in the microscopic image using a second machine learning model, the second machine learning model takes as input the unstained inference image, the generated virtual-stained image of sperm, and the quantity of sperm cells in the generated virtual-stained image, the second machine learning model trained using samples collected at known intervals post-coitus.

In yet another case of the system, the generator machine learning model generates two or more virtual-stained images of sperm, at least two of the virtual-stained images of sperm comprising different virtual stains.

In yet another case of the system, the one or more processors to further execute comprising a post-processing module to determine locations of sperm cells in the generated virtual-stained image of sperm by applying an intensity threshold across locations of the generated virtual-stained image of sperm, wherein the locations of sperm cells being where two or more of the stains are above the intensity threshold.

These and other embodiments are contemplated and described herein. It will be appreciated that the foregoing summary sets out representative aspects of systems and methods to assist skilled readers in understanding the following detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The features of the invention will become more apparent in the following detailed description in which reference is made to the appended drawings wherein:

FIG. 1 is a schematic diagram of a system for detection of sperm using virtual-staining, in accordance with an embodiment;

FIG. 2 is a flowchart of a method for detection of sperm using virtual-staining, in accordance with an embodiment;

FIG. 3 is a schematic of an example implementation of the system of FIG. 1;

FIG. 4 illustrates examples of intermediate output during training of the generator model in the system of FIG. 1;

FIG. 5 is a schematic of an example architecture of the generator model in the system of FIG. 1;

FIG. 6 is a schematic of an example architecture of the discriminator model in the system of FIG. 1;

FIG. 7A shows a fluorescence image collected from a representative mixture of sperm and cheek cells stained with DAPI and HY-LITER™ staining;

FIG. 7B shows an image generated by the generator model in the system of FIG. 1 when only the brightfield microscope image used in FIG. 7A (without staining) is provided as input;

FIG. 8 is an image showing virtual-colored output (DAPI and HY-LITER™) created by the generator model in the system of FIG. 1 using only brightfield microscope images of a model sample (in this example, buccal swab spiked with semen) as input overlaid with Xs of sperm identified by the system of FIG. 1;

FIG. 9 is a graph showing a count of sperm detected in dilutions of a mixture of sperm and cheek cells by the system of FIG. 1 in comparison to expected values as calculated from a known yield;

FIG. 10 illustrates a brightfield microscope image of a post-coital sample, overlaid with Xs of sperm identified by the system of FIG. 1;

FIG. 11 illustrates a chart of brightness correction to enable inference within training range for the system of FIG. 1;

FIG. 12 illustrates a representative comparison of paired HY-LITER™-stained and unstained post-coital sub-samples;

FIG. 13A illustrates a true fluorescence image collected from a representative mixture of sperm and cheek cells stained with DAPI and HY-LITER™;

FIG. 13B shows an output of the generator model in the system of FIG. 1 using a brightfield-equivalent of the image of FIG. 13A as input;

FIG. 14A shows a true fluorescence image collected from a representative mixture of sperm and cheek cells stained with DAPI and HY-LITER™.

FIG. 14B shows output of the generator model in the system of FIG. 1 using a brightfield-equivalent of the image of FIG. 14A as input;

FIG. 15 shows captured images of cells extracted from a vaginal swab collected 3 hours post coitus then stained using a HYLITER™ kit;

FIG. 16 shows a plot showing training progression in the presence of new input data; and

FIG. 17 shows captured images of cells extracted from a vaginal swab collected 24 hours post coitus then stained using HYLITER™ kit.

### DETAILED DESCRIPTION

Embodiments will now be described with reference to the figures. For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

Any module, unit, component, server, computer, terminal, engine or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors.

The following relates generally to microscopic image inspection, and more specifically, to a computer-based method and system for detection of sperm using virtual-staining.

In the present disclosure, “staining” refers to a chemical process whereby a contrasting agent (i.e., stain or dye) is added to a sample (e.g., a collection of biological cells) to enable better visualization, such as after microscope imaging. Additionally, in the present disclosure, “virtual-staining” refers to the production of generated image(s), from input image(s) capturing input sample(s), that appear to resemble stained input sample(s), even though the sample(s) were not actually stained.

The embodiments described herein provide an approach that advantageously removes both the need for sperm-staining, as well as the requirement of knowledge of sperm morphology. Advantageously, the approach described herein can automatically detect and quantify sperm within a complex sample. Embodiments of the present disclosure can be run in real time on a brightfield microscope, which are commonly available, and require minimal human preparation and activities; for example, only requiring that the specimen be dispensed from the sample swab onto a slide, sandwiched underneath a coverslip, and focused within the field of view on the microscope.

While the present disclosure generally describes use of bright field microscopy and phase contrast microscopy, it is understood that any suitable microscopy technique can be used; for example, phase contrast microscopy, dark field microscopy, differential interference contrast microscopy, fluorescence microscopy, computational phase imaging microscopy, and other computational microscopy approaches such as Fourier ptychographic microscopy and multi-spectral imaging. The present embodiments can be applied to commercially available microscopes or custom-built microscopes. The present embodiments can be standalone techniques or part of a larger system comprising other analyses approaches.

In brightfield microscopy, the background is illuminated, and objects in the light path attenuate the light, and thus, appear darker than the background. Therefore, the contrast mechanism that allows visualizing cells is the change in amplitude. In dark field microscopy, the background illumination is blocked in such a way that the background appears dark, and objects in the light path (at the focal plane) scatter the light, and thus appear brighter than the background. Therefore, the contrast mechanism that allows visualizing cells in dark field microscopy is also a change in amplitude. Light, as an electromagnetic wave, is generally described using both amplitude and phase. When light interacts with a material (e.g. sperm cell), both amplitude and phase change. Phase is generally not observable by the human eye or by general-purpose cameras, however, the changes in phase are more sample-dependent than changes in amplitude alone. In phase contrast microscopy, the background illumination is formed in a particular way using a ring, and the background illumination is dim. As the light passes through the object and its phase is modified, the microscope is built in a way such that this light that experienced phase changes constructively interferes with the background illumination. Thus, phase changes are translated to amplitude changes that are visible to the human eye and can be imaged. Importantly, the present inventors determined that sperm cells affect the phase much more strongly than non-sperm cells. Thus, sperm cells generally appear brighter than non-sperm cells in phase contrast microscopy.

While the present disclosure is generally directed to sperm detection for sexual assault forensic testing, it should be appreciated by a person skilled in the art that the embodiments described herein can be used in other areas; for example, in male fertility testing.

There are many stains that can be used for the detection and quantification of sperm cells within a sample. The present disclosure generally describes the use of the fluorescent HY-LITER™ staining kit. The antibody used in the HY-LITER™ kit has been determined to be highly specific for sperm cells. In this way, the HY-LITER™ kit serves as a suitable measure of ground truth for differentiating sperm from non-sperm cells within a heterogeneous mixture. While the fluorescent HY-LITER™ staining kit is generally used, any suitable stain can be used; for example, the haematoxylin and eosin (H&E) stain and the Christmas Tree (picroindigocarmine) stain. These two stains are generally easier to use than HY-LITER™ as they do not require fluorescence microscopy for readout; however, they are generally less specific than HY-LITER™ because they also stain other types of cells. This further staining presents an inherent problem to a naïve machine learning models that have no innate understanding of sperm morphology. Training such a model would require human annotation of the sperm to allow for learning of the sperm morphology; whereas training on HY-LITER™-stained cells generally requires no such annotation. In this way, with HY-LITER™-stained cells, training data can be acquired by simply receiving imaged slides; thus, generating brightfield (input) and fluorescence (ground-truth output) in a single imaging session, without the need for time-consuming human annotation. Staining by HY-LITER™ was verified by the present inventors to not have any impact on the brightfield image, which advantageously enables the capture of both input brightfield and ground truth fluorescence output in a single imaging session.

Whereas some other approaches use stains, such as the Christmas Tree stain, in conjunction with a machine learning models to perform automated sperm detection, the present embodiments advantageously provide that ability to perform sperm detection without staining with the use of in silico virtual-staining similar to the HY-LITER™ stain kit.

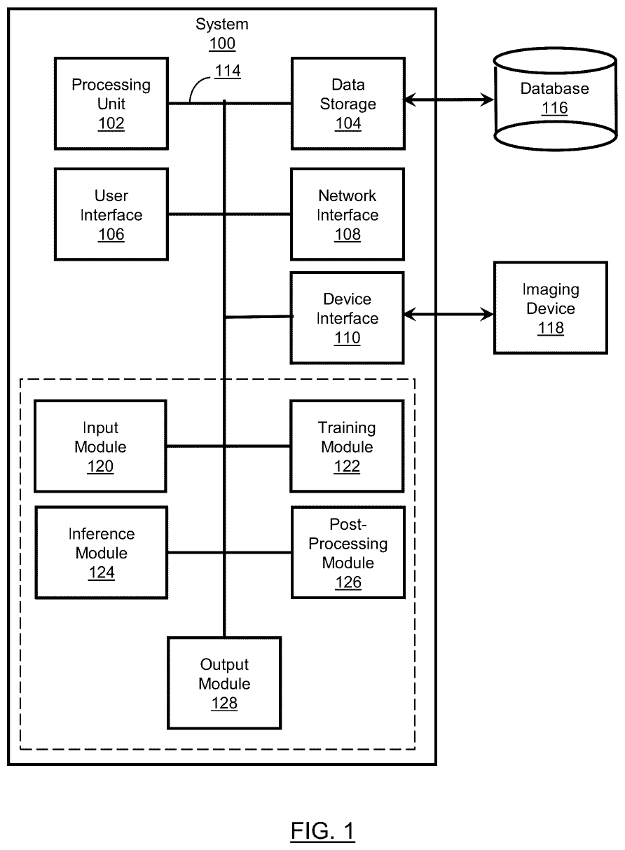

Referring now to FIG. 1, a system 100 for detection of sperm using virtual-staining, in accordance with an embodiment, is shown. The system 100 can be run on any suitable computing device, for example, on a local computing device, on a server, or the like. In some embodiments, the components of the system 100 are stored by and executed on a single computer system. In other embodiments, the components of the system 100 are distributed among two or more computer systems that may be locally or remotely distributed.

FIG. 1 shows various physical and logical components of an embodiment of the system 100. As shown, the system 100 has a number of physical and logical components, including a processing unit 102 (comprising one or more processors), data storage memory 104, a user interface 106, a network interface 108, non-volatile storage 112, and a local bus 114 enabling processing unit 102 to communicate with the other components. In some cases, the processing unit 102 can be a graphical processing unit. The processing unit 102 executes an operating system, and various modules, as described below in greater detail. The data storage memory provides responsive storage to the processing unit 102 and can be used to store the operating system and programs, including computer-executable instructions for implementing the operating system and modules, as well as any data used by these services. Additional stored data can be stored in a database 116. The user interface 106 enables an administrator or user to provide input via an input device, for example a keyboard and mouse. The user interface can also output information to output devices to the user, such as a display and/or speakers. The network interface 108 permits communication with other systems, such as other computing devices and servers remotely located from the system 100, such as for a typical cloud-based access model. In further cases, some operations of the processing unit 102 can be facilitated on a remote device; for example, operations can be performed on a remote server. The device interface 110 permits communication with an imaging device 118, for example, with a brightfield microscope, Fourier Ptychographic microscope, or other type of imaging device.

In an embodiment, the processing unit 102 can execute a number of conceptual modules, which can include an input module 120, a training module 122, an inference module 124, a post-processing module 126, and an output module 128. In some cases, the functions and/or operations of the conceptual modules can be combined or executed on other modules.

The system 100 can use both a machine learning model and a post-processing approach for sperm detection and quantification. In an example, the machine learning model can be based upon a generative adversarial network (GAN) architecture. The architecture of the GAN consists of two different networks that “compete” with one another during training. A generator network takes an input and produces a “fake” output. This output is taken alongside a set of real inputs (i.e., ground truth data) and fed into a discriminator network, which determines a “realness” of the output created by the generator network via comparison with the real ground-truth data. During the training loop, the generator learns to create outputs that are accurate, while the discriminator becomes better at determining which generated outputs are accurate. In this adversarial system, the networks compete to improve each other, and the discriminator effectively serves as a sophisticated loss function for the generator. Once training is completed, the generator network in the GAN can be used for inference. In this way, the trained GAN architecture can act like a convolutional neural network (CNN), such that it can be given an input (such as an image) and produce an output (such as a segmentation mask or fluorescence intensity).

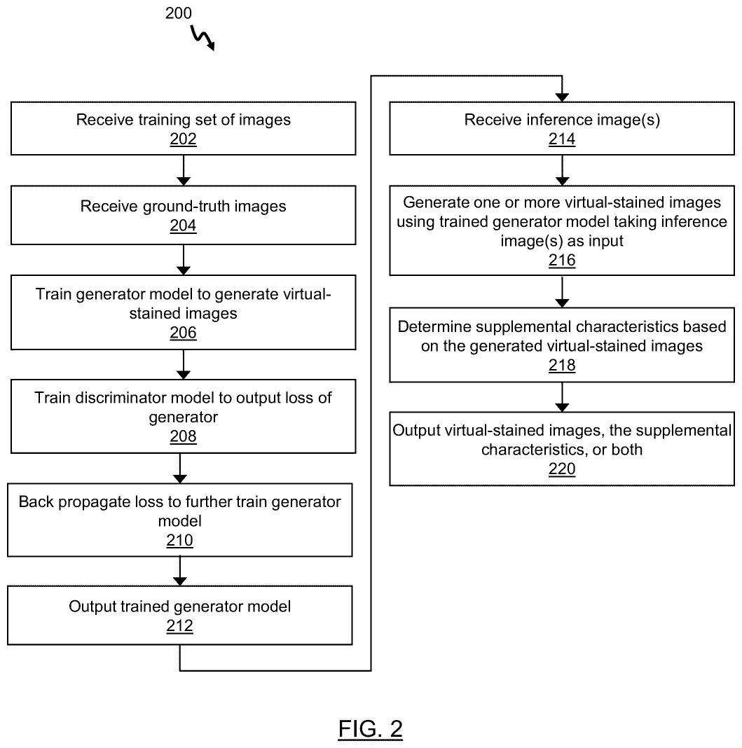

FIG. 2 illustrates a method 200 for detection of sperm using virtual-staining, in accordance with an embodiment. Blocks 202 to 212 represent a training phase while blocks 214 to 220 represent an inference or deployment phase.

At block 202, the input module 120 receives a set of training images comprising microscopic images of sperm cells; for example, as captured by a brightfield microscope. In some cases, the images can include images of sperm cells and epithelial cells. The set of training images can be split into training and test groups; for example, with an 80:20 split. While this example generally describes use of a brightfield microscope, any suitable imaging device can be used; for example, a Fourier Ptychographic microscope can be used to enable higher-resolution images from a fixed-position camera and/or objective.

At block 204, the input module 120 receives a set of ground-truth images that show staining of the sperm cells in the training images. Any suitable staining can be used, for example, DAPI stain, HY-LITER™ stain, haematoxylin and eosin (H&E) stain, Christmas Tree (picroindigocarmine) stain, or the like. In a particular case, the ground-truth images can include two or more types of staining of the training images; for example, HY-LITER™ fluorescence staining of the training images and DAPI (4′,6-diamidino-2-phenylindole) fluorescence staining of the training images. Generally, where two or more stains are used, ultimately resulting in two or more virtual-stains being generated, there is a substantial increase in accuracy and confidence of the output of the system 100.

At block 206, the training module 122 uses the training images and the ground-truth images to train a generator machine learning model to output generated virtual-stained images.

At block 208, the training module 122 uses the generated virtual-stained images outputted by the generator model and the ground-truth images to train a discriminator machine learning model to output a determination of a loss representing the accuracy of the generated virtual-stained images.

At block 210, the training module 122 back propagates the determined losses to the generator machine learning model for further training. In some cases, an optimizer can be used for backpropagation of calculated losses during training.

In a particular case, the generator loss, L_{tot,gen}, can be given by:

$${L}_{tot , gen} = {L}_{gan , gen} + λ {L}_{1} \qquad ( 1 )$$

where L₁ is an absolute difference between the generated image outputted by the generator model and a target image from the corresponding ground-truth image; i.e., the difference between a generated output fluorescence image and a ‘true’ fluorescence image. L_{gan,gen} is a binary crossentropy between an array of ‘True’ values (i.e., ground-truth values) and the sigmoidal probabilities produced by the discriminator model, which is essentially used in back propagation. Lambda is a parameterized value; for example, it can be chosen to be 100. In this way, the generator loss function can be a sum of the absolute difference of a target image compared to the generated image, and L_{tot,gen} reports the degree to which the generator has or has not ‘fooled’ the discriminator into producing high probabilities of Truth.

In a particular case, the discriminator architecture can have a convolutional neural network (CNN) architecture. In an example, the CNN can have a 25% dropout layer before a final layer, with leaky rectified linear unit intermediate activations, and a final sigmoidal activation to produce the output true/false probability.

Similar to Equation (1), in a particular case, the discriminator loss L_{tot,disc} can be as follows:

$${L}_{t o t , d i s c} = {L}_{gan , gen} + {L}_{r e a l} \qquad ( 2 )$$

where L_{gan,disc} is the binary crossentropy between an array of False values and the values produced by the discriminator model when given a generated input from the generator model. Lᵣₑₐₗ is the binary crossentropy between an array of True values and the values produced by the discriminator when given a real (i.e., ground-truth image) input. In this way, the discriminator learns to minimize the crossentropy for both real and generated inputs.

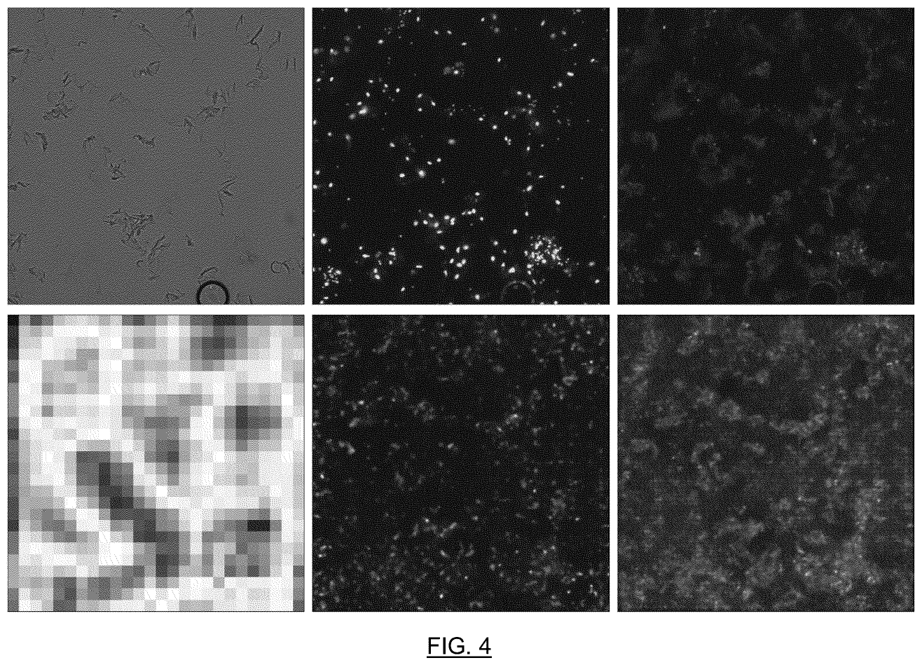

In a particular case, the optimizer can be an Adam optimizer to be used for the backpropagation of the calculated losses during training. In some cases, the optimizer can generally be formulated as a regression problem, where a mean squared error (MSE) can be used to monitor progress of the model during training such that the system can regress to correct fluorescence intensity values. In other cases, the optimizer can generally be formulated as a segmentation (per-pixel classification) problem, where categorical cross-entropy can be used to monitor the progress of the model during training; such as cases where HY-LITER™ values are binarized. In some cases, the generator model can be instructed to produce a series of randomly chosen outputs (e.g., 5) from the test set for every given number of epochs (e.g., 1000) in order to provide a means of determining an overfit of the generator model. In example experiments conducted by the present inventors, a sample of this progress can be seen in the output shown in FIG. 4, which has the true acquired brightfield, DAPI, and HY-LITER™ in the top row, with the bottom row being occupied by a “truth map” from the discriminator model, and the inferred DAPI and HY-LITER™ stains from the generator model. In some cases, monitoring of this changing output during training can allow for a means of interventional training. In the example experiments, after 37,000 epochs, a satisfactory convergence was found with a generator test set MSE value of 0.039.

In FIG. 4, from top left to right, shown are Brightfield, DAPI, and HY-LITER™ images, respectively, acquired from a representative mixture of sperm/cheek cells. From bottom left to right, shown are a “Truth map” from the discriminator model, an inferred DAPI image from the generator model, and an inferred HY-LITER™ image from the generator model. The truth map is a heat map ranging from high probability of truth to low probability of truth as output from the sigmoidal activation of the discriminator model.

At block 212, the training module 122 outputs the trained generator model as an excised generator network for use in the inference stage.

At block 214, the input module 120 receives an inference image(s) comprising a microscopic image comprising sperm cells; for example, as captured by a brightfield microscope.

At block 216, the inference module 124 provides the inference image as input into the trained generator model, the generator model generating a virtual-stained image of sperm from the inference image. In some cases, the generator model can generate two or more virtual-stained images representative of different stains; such as virtual-stained image similar to a HY-LITER™ fluorescent stain and a virtual-stained image similar to a DAPI fluorescent stain.

At block 218, in some cases, the post-processing module 126 can provide further processing on the generated virtual-stained image(s) to determine various supplemental characteristics. In a particular case, the post-processing module 126 can determine locations of sperm cells in the generated virtual-stained image of sperm by applying an intensity threshold across locations of the generated virtual-stained image of sperm. In further cases, the post-processing module 126 can determine a quantity of sperm cells in the generated virtual-stained image by determining contours around the locations having an intensity greater than the threshold, wherein each continuous region can be counted as a single sperm cell. In some cases, where the trained generator model outputted two or more virtual-stained images, the post-processing module 126 can perform a logical ‘AND’ of the virtual-stained images. Further, contours can be determined within an AND mask, for example, by using the OpenCV library. After contour determination, all continuous ‘True’ regions can be counted as a single sperm cell. In some cases, post-processing module 126 can further resolve overlapping continuous ‘True’ regions via application of mask erosion and dilation operations. In an example, the centers of these contours can be determined via calculation of the contour moments and division of the x and y moments by the contour mass; which can then be labeled accordingly.

At block 220, the output module 128 outputs the one or more virtual-stained images, the supplemental characteristics, or both, to the data storage 104, the database 116, the user interface 106, and/or the network interface 108. In some cases, the supplemental characteristics can be overlaid onto the outputted one or more virtual-stained images.

The present inventors conducted examples experiments to verify the accuracy and advantages of the present embodiments. In the example experiments, pure sperm samples (Caucasian ethnicity) and post-coital samples were acquired. Cheek cells were obtained via volunteer buccal swab. Once obtained, all samples were stored at −20° C. until thawed and used.

In the example experiments, for the sperm cell and cheek cell mixtures, the staining was performed in a tube before aliquoting the product onto a microscope slide and imaging. Briefly, a mixture of 5 μL 0.1 M DTT and 40 μL HY-LITER™ SP was formed. A 10 μL aliquot of sperm sample (of various densities) was added and mixed gently for 5 minutes. Then, 1 drop of HY-LITER™ blocking solution was added, and again mixed for 5 minutes before 1 drop of green HY-LITER™ stain solution was added. The mixture was then mixed via pipette for 5 minutes before being centrifuged for 3 minutes at 7,000 RPM. The supernatant was then removed before resuspending the pellet in 10 μL PBS (Phosphate Buffered Saline) for imaging.

In the example experiments, for the post-coital swab samples, the staining was performed as follows. ClickFit tubes were initially loaded with 400 μL PBS, then the sample swab was cut and inserted into the tube. The tube was vortexed for approximately 10 seconds, and then left to stand at room temperature for 5 minutes. The swab was then removed from the PBS and suspended above the PBS in a spin basket while being centrifuged at 13,000 RPM for 5 minutes. All but approximately 50 μL of supernatant was removed and discarded. The pellet was then resuspended in the residual supernatant via pipetting and vortexing. Once extracted, the post-coital samples were then stained in the tube using the HY-LITER™ approach. In some cases, post-coital samples were split, with one aliquot being stained (as above), while the other aliquot was processed identically except substituting all reagents in the HY-LITER™ kit with PBS.

After pre-processing of the samples in the example experiments, the samples were imaged by microscopy. A microscope slide was prepared by rinsing with ethanol and then drying at 56° C. for 5 minutes. The samples were then dispensed onto slides in 10 μL aliquots, and covered with HY-LITER™ mounting solution and a cover slip. Each sample was allowed to dry on a slide at room temperature, after which the mounting solution hardens and the sample and stains can be stable for months. Images were acquired using a tiling feature of the Nikon™ NIS Elements software via a 20× plan fluorescence DIC (0.5 NA) objective on a Nikon™ Eclipse Ni-E upright microscope. A PCO Edge 4.2 sCMOS camera was used for image capture, with an X-Cite™ Xylis™ light source employed for fluorescence imaging.

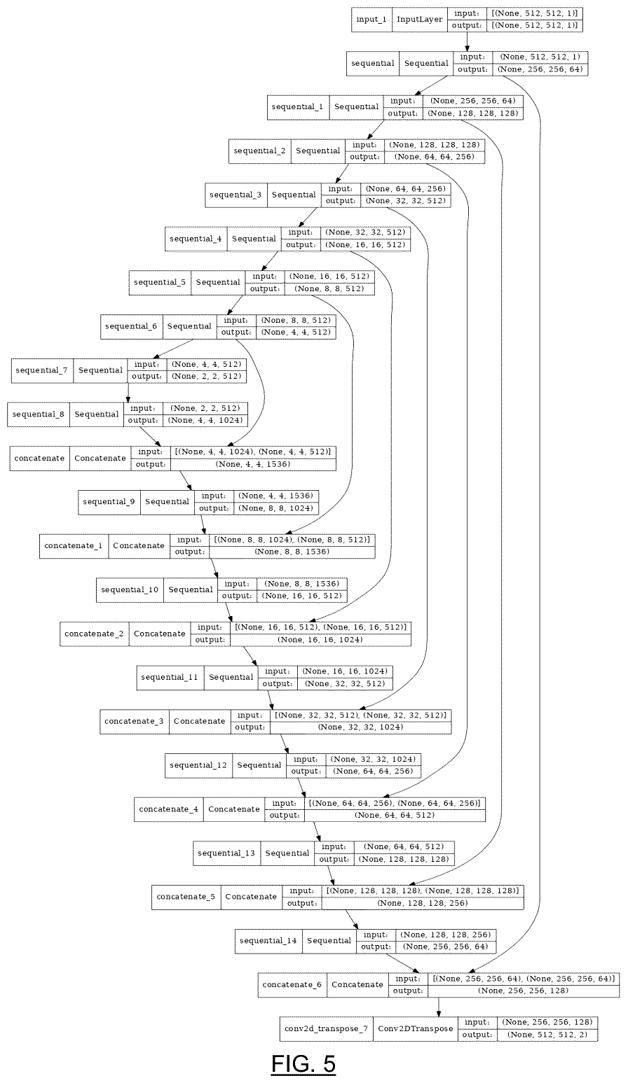

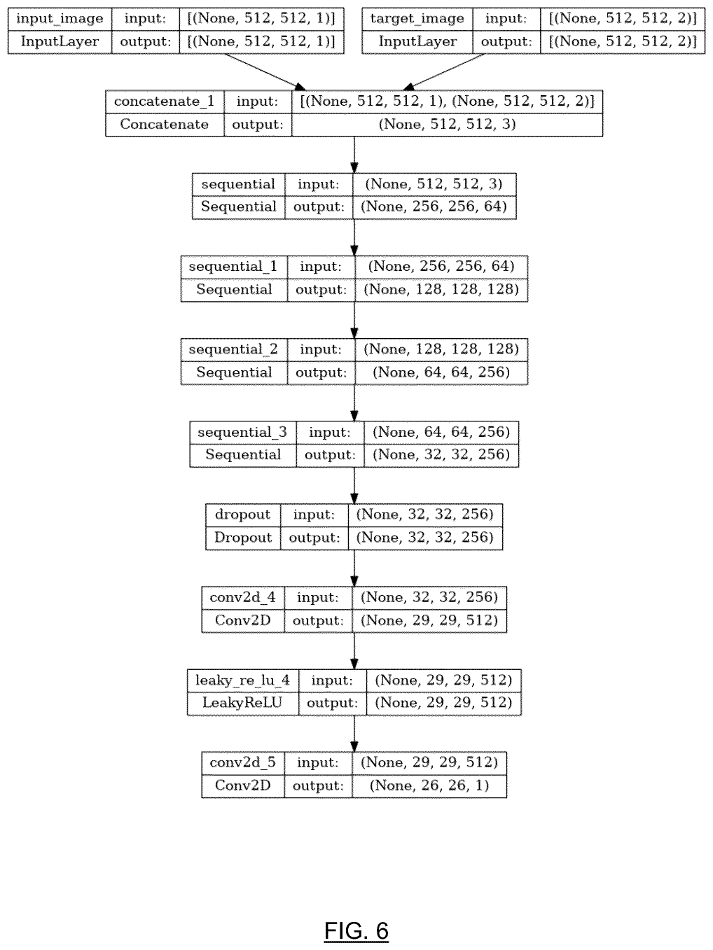

In the example experiments, the model employed canonical GAN architecture with a generator, discriminator and optimizer. For the generator, a U-Net architecture was chosen, with an input image size of 512×512 pixels, and a final output image of the same size, created by hyperbolic tangent functions on the final layer. Intermediate activation functions were rectified linear units, with some layers having 20% dropout. FIG. 5 shows a schematic of an example architecture of the U-Net generator for the GAN that was used for sperm cell identification. FIG. 6 shows a schematic of an example architecture of the discriminator that was used to determine values that correspond to perceived “real” or “fake” parts of the image created by the generator. In the example experiments, a total of 375 images were acquired and split 80:20 into training and test groups, with a fixed random seed of 42 for reproducibility.

It should be noted that the input images can be any suitable size, for example, 256×256 pixels. The smaller the input image size, generally the easier it is to run the described embodiments on lower-cost and/or more portable hardware. To enable compatibility, in some cases, larger images (for example 2044×2060 pixels) can be pre-processed. One approach for pre-processing involves resizing (e.g., compressing) the image to a selected smaller size (e.g., 512×512 pixels), which may lose some resolution. To preserve resolution, an approach can be used whereby the larger image is divided into smaller images, or tiles, of the selected smaller size. Images may also need to be padded, cropped, and/or resized before pre-processing to enable the tiling. For example, a 2044×2060 pixel (w×h) image may be modified by adding 4 pixels to the width and cropping 12 pixels from the height to convert the image to size 2048×2048, which would enable perfectly splitting the image into any of the following configurations (as examples):
An array of 2×2 tiles (total 4 tiles), each being 1024×1024;
An array of 4×4 tiles (total 16 tiles), each being 512×512;
An array of 8×8 tiles (total 64 tiles), each being 256×256;
An array of 16×16 tiles (total 256 tiles), each being 128×128;
and the like

The tiling approach described above can be performed the whole input image or the tiling can be informed by other pre-processing algorithms that may define tiles based on image properties (e.g., high contrast areas are in the middle of a tile). Generally, the pre-processing can be performed automatically programmatically; i.e., the user does not need to know the image size and/or define how much to pad and/or crop from the input image.

Additionally, the tiling approach described above can be performed in both the generator and the discriminator, both of which are described herein. In this way, the tiling approach can be used for both training and inference. After generating the tiles that make up the larger input image, the tiles can be combined to create an image with the same size as the original large input image.

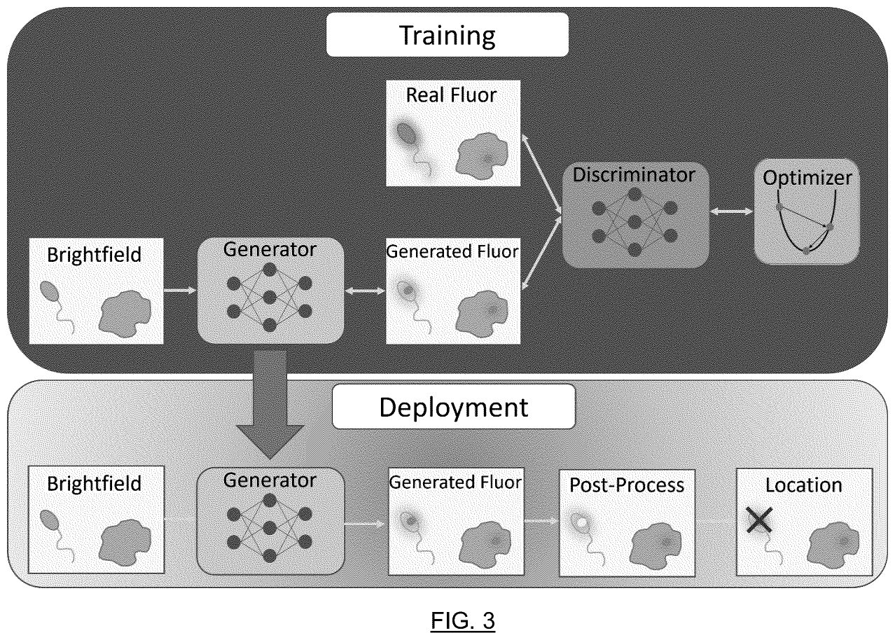

As illustrated in FIG. 3, in an implementation used in the example experiments, the GAN can be structured such that the inputs to the generator are brightfield images of sperm mixed with epithelial cells, and the ground truth was the HY-LITER™ and DAPI fluorescence staining of the same. This approach provides a generator capable of inferring the two stains solely from brightfield microscope input. Further post-processing can be performed to determine locations of the sperm and their count. Advantageously, the system 100 is capable of sperm quantification in one step after loading and imaging a sample in a brightfield microscope.

In the example output of FIG. 3, during training, the generator network can be fed only brightfield images, and produce “generated virtual fluorescence” images. These are compared with real fluorescence images via the discriminator network. The discriminator outputs values that correspond to perceived “real” or “fake” parts of the image created by the generator. The optimizer takes the output of the discriminator, calculates losses corresponding to how well each network performed, and then uses them for back-propagation. In deployment, the output of the generator can be used such that final sperm locations can be found. Further advantageously, post-processing can determine a final count of detected sperm based on brightness-corrected continuous regions in an intersection between two or more different generated virtual-stains (for example, HY-LITER™ and DAPI virtual-stains).

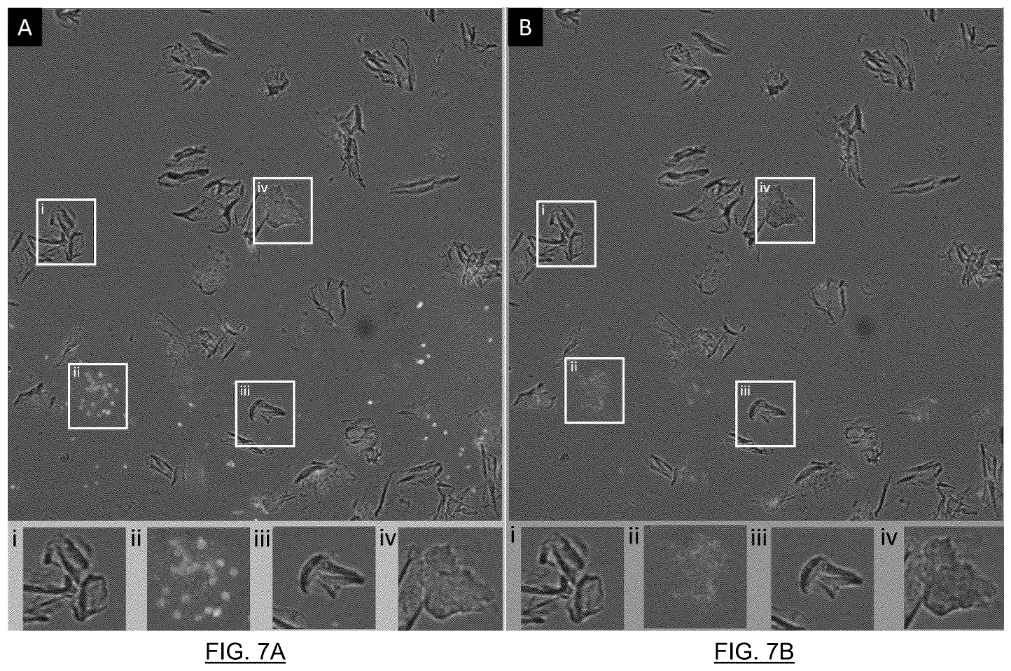

FIGS. 7A and 7B show a comparison of a real fluorescence image with an image outputted by the system 100. FIG. 7A shows a fluorescence image collected from a representative mixture of sperm/cheek cells stained with DAPI and HY-LITER™ staining. FIG. 7B shows an image generated by the generator model when only the brightfield microscope image used in FIG. 7A (without staining) is provided as input. The outputted image represents an inference of fluorescence that was generated by passing a brightfield microscope image through the generator model. As can be seen in FIGS. 7A and 7B, the images are substantially similar. As illustrated in the insets, the generator model appears to appropriately show the DAPI intensities, which signifies correlation between brightfield morphologies and the DAPI stain.

As seen in FIGS. 7A and 7B, the generated fluorescence images advantageously lack major optical and staining aberrations. In inset iv, there appears to be a large area of some object has been stained by DAPI, but this area is absent in the image generated by the system 100. This desirable effect likely arises from the fact that the model has learned from the training dataset that DAPI stains typically occupy a small, circular region, and not often a large non-circular region, like the one shown in the center of FIG. 7A. The generator model appears to reject large areas of aberrant fluorescence, as they are unusual (i.e., appear very few times in the training data) and also have no apparent indicators for their presence in the brightfield images.

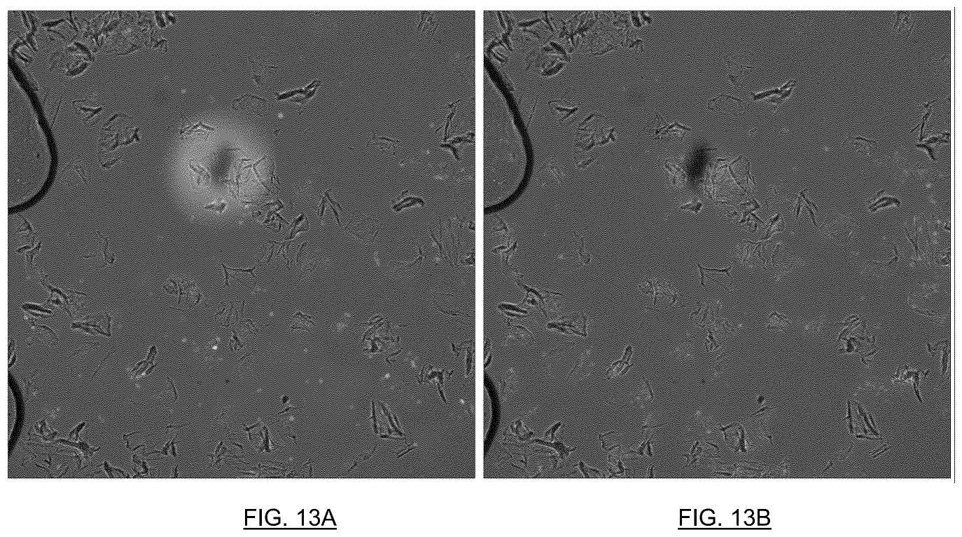

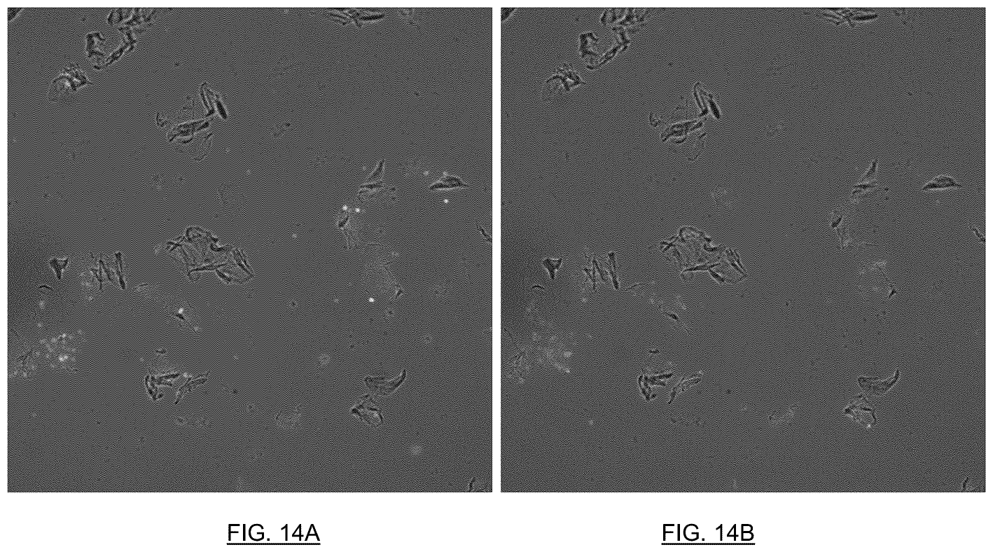

The above advantages are also illustrated in FIGS. 13A, 13B, 14A, and 14B. FIGS. 13A and 13B shows sample output showing aberrant fluorescence rejection by the generator model. FIG. 13A illustrates true fluorescence image collected from a representative mixture of sperm and cheek cells stained with DAPI and HY-LITER™. FIG. 13B shows the output of the generator model using a brightfield-equivalent of the image of FIG. 13A as input. FIGS. 14A and 14B show another example of aberrance rejection. FIG. 14A shows a true fluorescence image collected from a representative mixture of sperm and cheek cells stained with DAPI and HY-LITER™. FIG. 14B shows output of the generator model using a brightfield-equivalent of the image of FIG. 14A as input.

In some cases, after the virtual-stained fluorescence image was generated by the generator model, supplemental characteristics can be determined. For example, a threshold and a logical AND can be used to yield precise locations of the sperm cells. The threshold used for binarization of the image was chosen to be 90% of the max intensity value. This value was chosen because it caused very few regions to “bleed” into one another and yielded the closest value to expected results. Other image processing techniques can be used, such as erosion, but it was found that the 90% threshold technique yielded similar results with less computation time and prevented the phenomenon observed for erosion in which very small regions of high intensity can be erroneously removed.

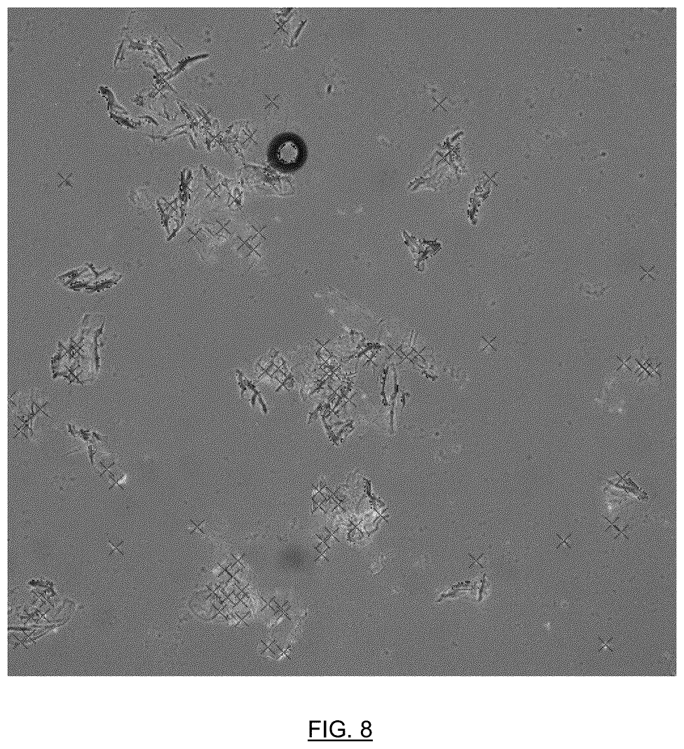

In some cases, once the threshold is applied, the contours of the remaining binary image can be obtained, and each continuous region can be counted as a single sperm cell in order to determine a quantity of sperm cells in the generated image. FIG. 8 illustrates centers of the continuous regions denoted with an “X”. FIG. 8 is an image showing the virtual-colored output (DAPI and HY-LITER™) created by the generator model using only brightfield microscope images as input, the input having a mixture of sperm and cheek cells therein. X-marks indicate each location that the post-processing module 126 located a sperm cell. The X-marks signify a single continuous region where both DAPI and HY-LITER™ are present in high intensity.

In addition to the above, a further supplemental characteristic was determined by summing the number of these regions, yielding a quantity of sperm predicted to be in the sample. Ultimately, the system 100 advantageously allows for a one-step sample-to-result workflow, wherein the user can simply insert the sample slide and get a number of characteristics, such as the quantity of sperm within the sample. At the same time, if the user is suspicious of the results, or if there is a noticeable visual abnormality within the sample, the user can look at the virtual-stained generated output to get a sense of what objects are being labeled as sperm cells. Additionally, the threshold and/or erosion operations can be adjusted to increase or decrease the stringency of the detection; which could be useful in case of samples that have been extremely diluted.

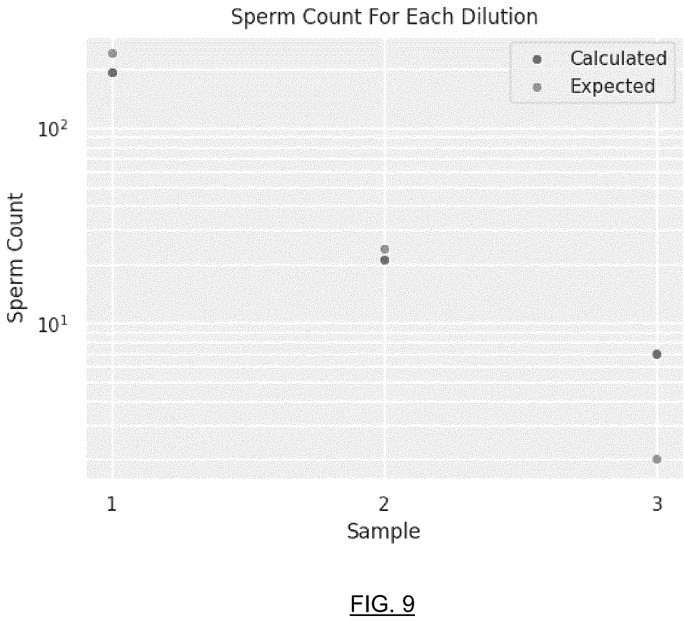

To examine the accuracy of the generator model and post processing, the example experiments used a set of serial 10× dilutions, wherein sperm concentrations were diluted while being mixed with cheek cells of a fixed concentration. The results of this experiment can be seen in the chart of FIG. 9 and TABLE 1, below. FIG. 9 shows quantification of sperm dilutions as a graph; particularly, the count of sperm detected in dilutions of a mixture of sperm and cheek cells by the system 100 in comparison to the expected values as calculated from known yield. Sample 1 is the highest concentration, with samples 2 and 3 being serial 10× dilutions of sample 1. TABLE 1 shows the calculated values for these serial dilutions based upon the post-processing calculations and predetermined expected values.

TABLE 1

| | |
|---|---|
| Sample | Sperm Count |

| Number | Calculated | Expected |
|---|---|---|
| 1 | 191 | 240 |
| 2 | 21 | 24 |
| 3 | 7 | 2 |

As indicated, the system 100 outputs sperm quantities that are close to the expected values in the artificial sperm and cheek cell samples. Additionally, the 10× dilution quantities are noticeably linear when plotted on a log scale, suggesting that the generator and post processing are accurate across a wide range of sperm concentrations. In some cases, because accuracy generally drops when the sperm count is near zero, when used in a critical application, a minimum value threshold can be used. In practice, samples at such a low concentration are often carefully processed by technicians anyway, as the low amount of genetic material necessitates careful handling so that downstream processes may be utilized as well.

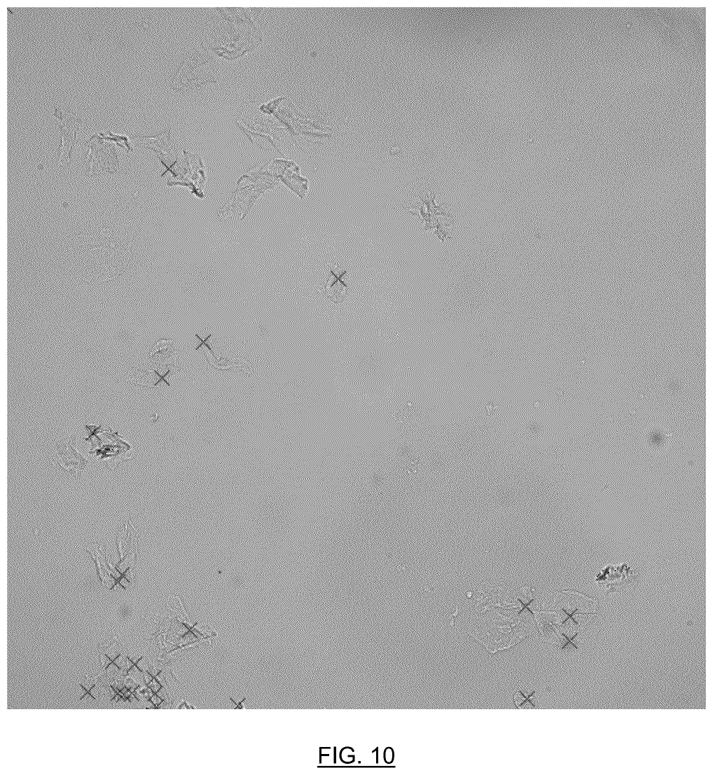

The example experiments also applied the system 100 to “real-world” samples that were collected with a swab post-coitus. A representative result for this type of sample is shown in FIG. 10. FIG. 10 shows brightfield microscope image of a post-coital sample, overlaid with Xs of sperm identified by the system 100. FIG. 10 can be representative of what the system 100 can output to a user at the point of care, such as to a Sexual Assault Nurse Examiner (SANE).

As illustrated in FIG. 10, 21 sperm cells were identified from the brightfield by the system 100, which were subsequently confirmed manually by inspecting the HY-LITER™-stained image. In the example experiments, the output image was generated in a short period of time of approximately five minutes; which included extracting from the swab, resuspending, dispensing onto the slide, drying, and imaging.

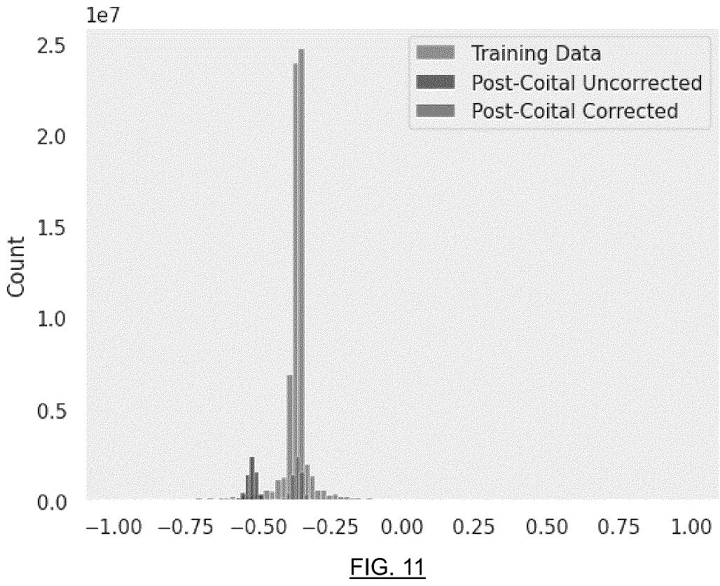

In some cases, the input module 120 and/or post-processing module 126 can also perform brightness-correction. For example, for instances where the inference image was collected with reduced brightness (with smaller shutter aperture in illumination path in the microscope). Even if such differences may not be perceptible for a human observer by eye, if not corrected, it can result in inaccurate outputs when used as input for the generator model; particularly because the pixel intensity values may be out of the range presented in the training data. Thus, brightness correction can be used to automatically adjust each image such that it can be reliably analyzed by the system 100; as illustrated in the chart of FIG. 11. FIG. 11 shows a histogram illustrating pixel intensity values normalized to the range [−1, 1]. Without correction, the post-coital sample has much lower intensity values than what would be expected. When the brightness values are shifted into the same range as the training data, the resulting corrected output results in more reliable data for use in inference by the generator model.

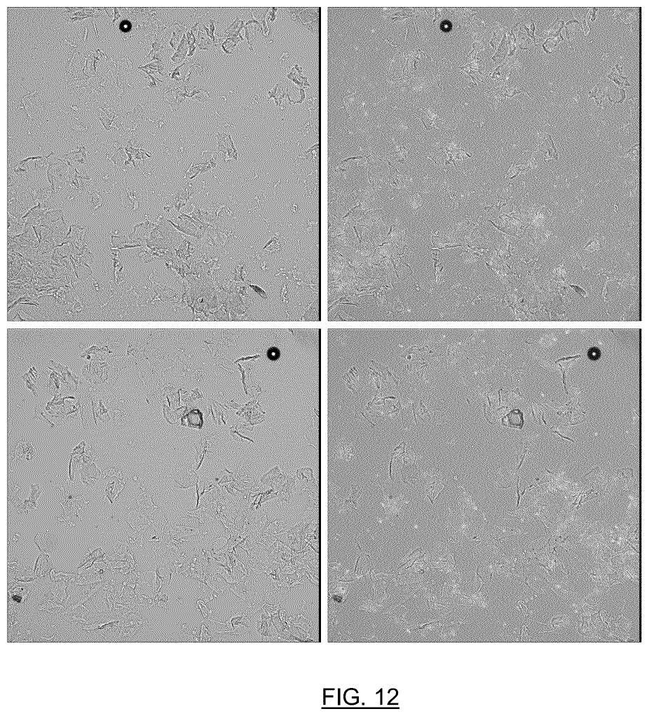

The example experiments also evaluated post-coital samples that were unstained. This experiment was designed to evaluate whether the system 100 might be identifying sperm cells via a possible low fluorescence signal that “leaks” through to the brightfield (though it is imperceptible to a human observer). Post-coital samples were split into pairs of HY-LITER™-processed sub-samples and sub-samples in which all of the HY-LITER™ _workflow reagents were replaced with PBS. The results are illustrated in FIG. 12. As shown, the results are substantially similar. Additionally, the post-processing module 126 was used to count the sperm cells as the paired aliquots. The quantities for the images in FIG. 12 were 459 sperm cells per microliter for the virtual-stained sample, and 442 sperm cells per microliter for the unstained split of the same sample; constituting a difference of approximately 4%. These results illustrate that there is minimal “bleed-through” effect on the system 100, such that the claimed approach can be used without requiring staining.

FIG. 12 illustrates a representative comparison of paired HY-LITER™ stained/unstained post-coital sub-samples. The upper left image shows a brightfield image of HY-LITER™-stained sub-sample. The upper right image shows an inferred fluorescence generated by the system 100, using the upper-left image as input, overlaid on the original brightfield image. The lower left image shows a brightfield image of an unstained sub-sample. The lower right image shows an inferred fluorescence generated by the system 100, using the lower-left image as input, overlaid on the original brightfield image.

Advantageously, using only a brightfield microscope input image, the system 100 is able to determine sperm location and quantities; thus, enabling users without expert knowledge of sperm staining and morphology to effectively determine the presence and amount of sperm within a sample. This provides an important breakthrough as a technician on-site can determine whether there is sperm present; where, typically, this information is often not known until weeks or months after sample collection. In further cases, the system 100 can be used for sperm DNA acquisition from forensic samples; for example, utilized in combination with a digital microfluidic isolation of single cells for Omics (DISCO). Specifically, because the sperm are precisely located and adhered to the surface, they should be amenable to laser-lysis and collection of their contents on the DISCO platform. Advantageously, this could result in an approach where sperm are not only detected, but their DNA is acquired and shuttled to downstream processing on-chip in a completely automated fashion. Employing such an approach in a hospital setting could enable DNA identification of sexual assault perpetrators within a matter of hours or even minutes after the sample is acquired. The present embodiments allow users, such as SANEs, to process sexual assault samples at the point-of-care and therefore lighten the workload of forensic technicians; as well as bolster confidence in the justice system for those that have been victimized.

The embodiments described herein advantageously allow for sperm quantification by non-expert users, such as nurses. By lessening the difficulty of this task, the workload of forensic scientists will be reduced, and the processing of sexual assault kits can be performed quicker; such as at the same location that they were acquired. This, in turn, will lower reporting barriers for the victims of sexual assault and foster confidence in the handling of sexual assault cases and the justice system overall. Victims groups have indicated that if a rapid protocol could be implemented early in the process, a much larger percentage of assault victims might be willing to move forward to press charges as the victim leaves the SANE better informed about the evidence collected.

Further, the present embodiments do not use any stain, and therefore, there are no negative impacts for downstream analysis; such as for DNA analysis. This is particularly beneficial where there is a limited quantity of sample and a forensics lab would also like to perform short tandem repeat (STR) analysis on the DNA in the sample to determine an identity of the assailant; which is generally not possible if the sample is stained.

In some cases of the present embodiments, by generating a virtual-stained image with two stains, the intersection between these two virtual stains can be used to provide enhanced and accurate detection of sperm cells.

In some cases, the system 100 can be implemented in a small, self-contained device that accepts a microscope slide that has been smeared with the tip of a vaginal swab sample, prior to a cover slip being added to the slide. After the slide sample has been inserted to the imaging device 118, the system 100 can generate a report of the quantity of sperm detected within the sample, as well as the precise locations of the sperm detected within the smeared sample. This report could then be communicated to a user device or displayed on the user interface 106 for assessment. In some cases, this implementation can be battery-powered, such that it could be deployed at the point of care (e.g., in an ambulance at the reported site of sexual assault).

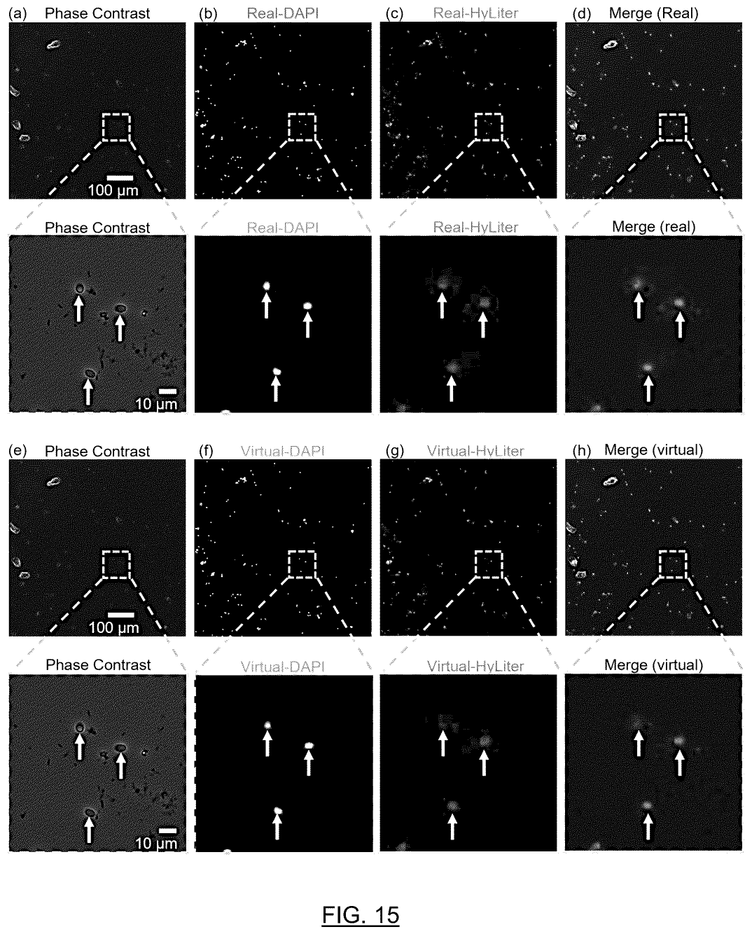

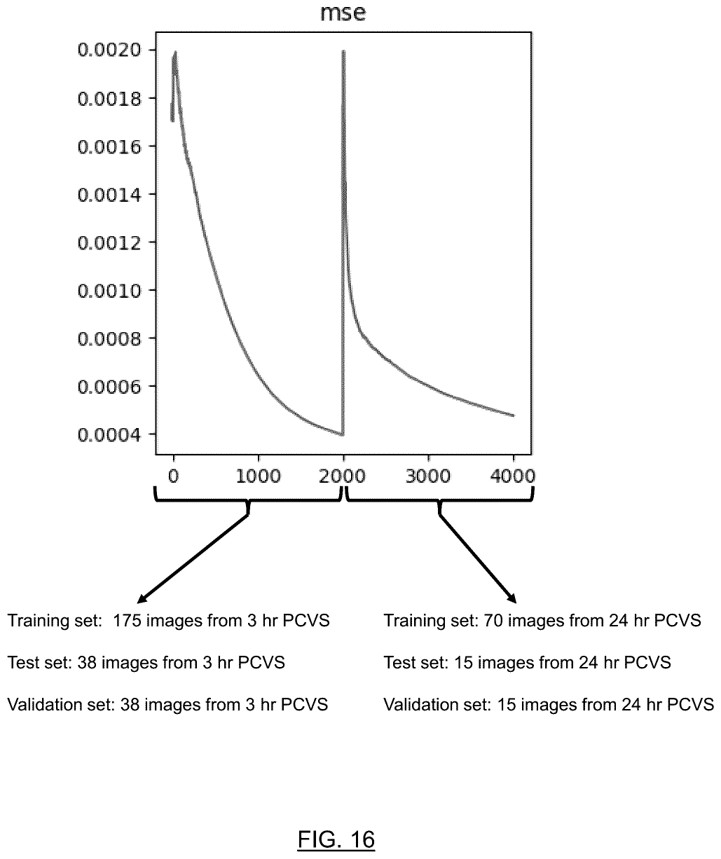

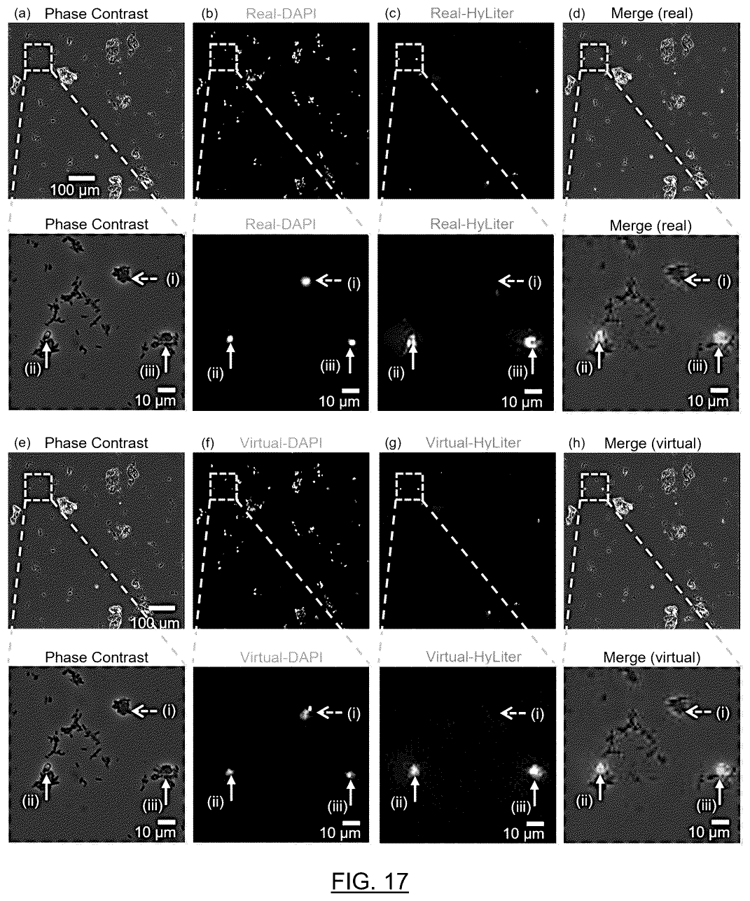

In the example experiments, the present inventors also investigated the performance of the system 100 when the input image is obtained using phase contrast microscopy. FIG. 15 illustrates results from the example experiments where the input image is captured using phase contrast microscopy. Because sperm are more easily discernible from non-sperm cells when observed using phase contrast microscopy, the example experiments illustrate that the model of the present embodiments performs better than with input images captured using bright field microscopy. Only 2000 epochs were needed to achieve a mean squared error of 0.0004, when trained with images of cells extracted from a vaginal swab collected 3 hours post-coitus. When the model was applied to images of cells extracted from a vaginal swab collected 24 hours post-coitus, the MSE was 0.002. An additional 2000 epochs of training using images from cells extracted from the vaginal swab collected 24 hours post-coitus recovered the performance closer to the 3 hour post-coitus results, with an MSE of 0.0005; as illustrated in FIG. 16. FIG. 17 shows exemplary results obtained for samples examined 24 hours post-coitus. These results illustrate that the system 100 can continuously improve as the model is trained with more samples. It also illustrates that the system 100 generalizes to samples and lighting conditions that the model may have not seen during training.

FIG. 15 shows images of cells extracted from a vaginal swab collected 3 hours post coitus then stained using a HYLITER kit (DAPI and Alexa488 fluorophores). Image (a) shows phase contrast microscopy, image (b) shows a real DAPI channel, and image (c) shows a real Alexa488 channel. Image (d) shows a merger of images (a), (b) and (c). Insets in images (a) to (d) are a close-up depicting 3 sperm channels, marked with vertical arrows with solid lines. Image (e) is a duplicate of image (a) for illustrative purposes. Image (f) is a virtual-DAPI image generated by the system 100 showing very close resemblance to the real-DAPI image (b). Image (g) is a virtual-Alexa488 generated by the system 100 showing very close resemblance to the real-Alexa488 image (c). Image (h) is a merger of images (e), (f) and (g), showing a very close resemblance to image (d).

FIG. 16 illustrates a plot showing training progression in the example experiments. The left side of the plot shows when images from the 3 hr PCVS samples were used. At epoch 2000, the model was used to generate the images shown in images (f) and (g) of FIG. 15.

Subsequently, for epoch 2001, images from 24 hr PCVS samples were used, showing a higher error initially. After the model trained for another 2000 epochs, the MSE converged to more useable results (prototypical generated images shown in images (f) and (g) of FIG. 17).

FIG. 17 shows images of cells extracted from a vaginal swab collected 24 hours post coitus then stained using a HY-LITER™ kit (DAPI and Alexa488 fluorophores). Image (b) shows a real DAPI channel and image (c) shows a real Alexa488 channel. Image (d) is a merger of images (a), (b) and (c). Insets in images (a) to (d) show close-up images. Horizontal arrow with dashed line points to (i) non-sperm cells that appear bright under the DAPI channel (nuclear stain) and dark under the Alexa488 channel (sperm-specific). Vertical arrows with solid line point to sperm cells that appear bright under the DAPI channel (nuclear stain) and bright under the HY-LITER™ channel (sperm-specific). For arrow (iii), even though the sperm cells looks like it's underneath a non-sperm cell, the fluorescent signal in the Alexa488 channel enables identifying sperm cells. Image (e) is a duplicate of image (a) for illustrative purposes. Image (f) is a virtual-DAPI image generated by the system 100 that very closely resembles the real-DAPI image (b). Image (g) is a virtual-Alexa488 image generated by the system 100 that very closely resembles the real-Alexa488 image (c). Image (h) is a merger of images (e), (f) and (g) that very closely resembles image (d). Insets in (e) to (h) show close-up images. Horizontal arrow with dashed line points to (i) non-sperm cells that appear bright under the virtual-DAPI channel (nuclear stain) and dark under the virtual-Alexa488 channel (sperm-specific). Vertical arrows with solid line point to sperm cells that appear bright under the virtual-DAPI channel (nuclear stain) and bright under the virtual-HY-LITER™ channel (sperm-specific). For arrow (iii), even though the sperm cells looks like it's underneath a non-sperm cell, the virtual-Alexa488 channel enables identifying sperm cells.

While the example experiments generally describe additional training using only the newly received data, the system 100 can also proceed by combining the older training data with the newly received training data, and re-training of the model. Although this approach may be more time-consuming (because the training set can be quite large), such approach can ensure that the model performs reasonably well for samples that are similar to the samples used in the initial training.

Advantageously, the example experiments with new training data illustrates that the system 100 is able to handle advances in technology. For example, there are different variations of the HY-LITER™ stain; one for staining on slide (referred to as ‘HY-LITER™ Express’) and another for staining in solution (referred to as ‘HY-LITER™ SOS’). Additionally, the performance of HY-LITER™ staining has been improved by making changes to the manufacturer's protocol (e.g., using 1 M DTT instead of 0.1 M DTT). It can be expected that additional protocol improvements and different variations to the staining kits will be arrived at over time, for example, using a CF488A fluorophore instead of Alexa488 fluorophore. A further variation of the HY-Liter™ kit can use other propidium iodide (PI) instead of DAPI as the nuclear stain (thus, not requiring exposure to UV light). The system's 100 ability to perform well to newly inputted data shows that the model, and thus the system 100, would still be useable in such situations and variations.

Generally, it is critical to virtual-stain non-sperm cells correctly, even more so than virtual-staining sperm cells correctly. In this way, non-sperm cells should have high intensity pixels in the virtual DAPI channel but not in the virtual HY-LITER™ channel.

In practical circumstances, the amount of sperm cells found on an input sample (i.e., slide) is generally used for several purposes: to establish a timeline (e.g., this amount of sperm detected and/or a ratio of sperm to non-sperm cells is consistent with sperm being deposited x hours prior to sample collection), and to inform downstream processing (e.g., if lots of sperm is detected, some approaches would work, and otherwise, other approaches should be used).

While the present disclosure generally describes a determination of how sperm is present in a given sample, it should be understood that the present embodiments can likewise, or additionally, be used to determine how many non-sperm cells are present in a given sample. Particularly, by determining areas that are relatively high in the DAPI channel and not in the virtual HY-LITER™ channel (also referred to as the ‘Alexa488 channel’). In some cases, other constraints may be used to limit an artificially high count (e.g. circularity, size, or the like).

In some cases, the post-processing module 126 can use a second machine learning model downstream of the virtual staining model, and/or sperm or non-sperm cell counting, to generate further insights. In an example, the second model can take as input one or more of: (i) the original input sample image, (ii) the virtual stain images, and (iii) the sperm and/or non-sperm counts, and output an estimate of a time interval between sperm deposition and sample collection. This example can either be implemented using a regression model or a classification model. In the case of the latter, the model can be trained with various classes, with each class containing samples collected at certain time points. For example:
Class 0: no sperm (vaginal sample collected after 2 weeks of abstinence);
Class 1: 0 to 3 hours PC (vaginal sample collected 0 to 3 hours post-coitus);
Class 2: 3 to 6 hours PC (vaginal samples collected 3 to 6 hours post-coitus);
Class 3: 6 to 12 hours PC (vaginal samples collected 6 to 12 hours post-coitus);
Class 4: 12 to 24 hours PC (vaginal samples collected 12 to 24 hours post-coitus); and
Class 5: 24 to 48 hours PC (vaginal samples collected 24 to 48 hours post-coitus).

While the present disclosure generally describes input data that has been stained with the HY-LITER™ kit (DAPI+Alexa488 fluorophores) that takes one image as input and generates two images (virtual DAPI and virtual Alexa488 channels), other suitable staining kits can be used. For example:
A Christmas tree stain that takes one image as input (could be monochrome or could be color, composed of three channels R, G, B) and generates one image with three channels, one for each of R, G and B;
A Hematoxylin & Eosin stain that takes one image as input (could be monochrome or could be color, composed of three channels R, G, B) and generates one image with three channels, one for each of R, G and B.

In further cases, a single model can be used that takes as training input a combination of images using different stains. For example, the training input images can include images each having one of the HY-LITER™ stain, the Christmas tree stain, and the Hematoxylin & Eosin stain. In this way, the model can take a single image as input and generate three image sets: one for HY-LITER™ (2 images: virtual-DAPI and virtual-Alexa488), one for Christmas tree stain (3 channels: virtual-R, virtual-G, virtual-B) and one for Hematoxylin & Eosin (3 channels: virtual-R, virtual-G, virtual-B). To reduce training requirements, transfer learning may be employed. Additionally, such model can be used without staining or with staining. While the most useful use case is without staining, forensic labs may be interested in trying to stain the same sample with more than one stain, which may be infeasible. Using such a model, the forensic lab technician can perform the stain that they are familiar with and produce the other virtual stains.

While the present embodiments generally describe use of stains, it is understood that other forms of microscopy determinations can be used. For example, for fluorescence microscopy, a cell's autofluorescence can be used such that the model takes a fluorescent image, with unstained cells, and generate images as if the input image was stained with any of the above stains described herein (e.g., a virtual DAPI and virtual HY-LITER™, or virtual-R, virtual-G, and virtual-B pertaining to a virtual Christmas tree stain, or a virtual H&E stain, etc.).

While mean squared error was generally used in the example experiments as a metric to evaluate the performance of the system 100, it should be understood that any other metrics may be used, such as but not limited to mean logarithmic squared error and intersection over union.

While the present disclosure generally describes sperm detection for sexual assault processing, it should be understood that the present embodiments can be used in any field where sperm is to be detected from a sample; such as for fertility determinations. In some cases, the determinations can be combined with other tests, such as with human leukocyte antigen (HLA) testing.

The computational complexity and data processing requirements of the described embodiments significantly exceed what could be performed effectively through human mental processes, underscoring the technical nature and necessity of computer implementation. The invention provides a solution to generating virtually stained images of sperm, which necessitates machine learning models and techniques that process vast amounts of data with high efficiency and accuracy. In some cases, the described embodiments leverage advanced and sophisticated mathematical models and techniques that surpass human cognitive capabilities in speed, accuracy, and scalability, in order to achieve the technical advancements. The interaction between these components/steps and the technical outcomes achieved by the present embodiments are impractical for human cognition to replicate due to the scale and complexity of the operations involved. In some cases, there is increased reliability and robustness due to virtual staining that leverages machine learning models that generate outputs and optimize such outputs in ways beyond human cognitive capabilities. The entire disclosures of all references recited above are incorporated herein by reference.

Although the invention has been described with reference to certain specific embodiments, various modifications thereof will be apparent to those skilled in the art without departing from the spirit and scope of the invention as outlined in the claims appended hereto.

1. A computer-implemented method for detection of sperm, the method comprising:
receiving an unstained inference image, the inference image comprising a microscopic image capturing sperm cells;
generating a virtual-stained image of sperm from the inference image using a trained generator machine learning model, the generator machine learning model taking the inference image as input, the generator machine learning model trained using a set of training images comprising microscopic images of sperm cells and a set of ground-truth images showing staining that identifies the sperm cells in the training images, the generator machine learning model trained by propagating determined losses between generated virtual-stained images and corresponding ground-truth images; and
outputting the generated virtual-stained image of sperm.

2. The method of claim 1, wherein the staining that identifies the sperm cells in the ground-truth images comprises staining of only the sperm cells.

3. The method of claim 1, the staining that identifies the sperm cells in the ground-truth images comprises at least two different types of stains.

4. The method of claim 1, wherein the generator machine learning model is trained using a first set of training images comprising microscopic images of sperm cells collected at a first time-period post-coitus and a first set of ground-truth images showing staining that identifies the sperm cells in the first set of training images, and wherein the generator machine learning model is further trained using a second set of training images comprising microscopic images of sperm cells collected at a later time-period post-coitus and a second set of ground-truth images showing staining that identifies the sperm cells in the second set of training images.

5. The method of claim 1, further comprising determining locations of sperm cells in the generated virtual-stained image of sperm by applying an intensity threshold across locations of the generated virtual-stained image of sperm.

6. The method of claim 5, further comprising determining a quantity of sperm cells in the generated virtual-stained image by determining contours around the locations having an intensity greater than the threshold, wherein each continuous region can be counted as a single sperm cell.

7. The method of claim 1, further comprising determining a time interval between sperm deposition and sample collection of the sperm cells captured in the microscopic image using a second machine learning model, the second machine learning model takes as input the unstained inference image, one or more generated virtual-stained images of sperm, and the quantity of sperm cells in the one or more generated virtual-stained images, the second machine learning model trained using samples collected at known intervals post-coitus.

8. The method of claim 1, wherein the generator machine learning model generates two or more virtual-stained images of sperm, at least two of the virtual-stained images of sperm comprising different virtual stains.

9. The method of claim 8, further comprising determining locations of sperm cells in the generated virtual-stained image of sperm by applying an intensity threshold across locations of the generated virtual-stained image of sperm, wherein the locations of sperm cells being where two or more of the stains are above the intensity threshold.

10. The method of claim 1, wherein the generated virtual-stained image of sperm comprises one or more of virtual HY-LITER fluorescent staining, virtual DAPI (4′,6-diamidino-2-phenylindole) fluorescent staining, virtual haematoxylin and eosin staining, and a virtual picroindigocarmine staining.

11. The method of claim 1, further comprising performing pre-processing on the unstained inference image, the pre-processing comprising dividing the inference image into tiles and providing each of the tiles as input to the generator machine learning model.

12. A system for detection of sperm, the system comprising one or more processors in communication with a data storage memory, the data storage memory comprising instructions for the one or more processors to execute:
an input module to receive an unstained inference image, the inference image comprising a microscopic image capturing sperm cells;
a training module to train a generator machine learning model using a set of training images comprising microscopic images of sperm cells and a set of ground-truth images showing staining of the sperm cells in the training images, the generator machine learning model trained by propagating determined losses between generated virtual-stained images and corresponding ground-truth images;
an inference module to generate a virtual-stained image of sperm from the inference image using the trained generator machine learning model, the generator machine learning model taking the inference image as input; and
an output module to output the generated virtual-stained image of sperm.

13. The system of claim 12, wherein the staining that identifies the sperm cells in the ground-truth images comprises staining of only the sperm cells.

14. The system of claim 12, the staining that identifies the sperm cells in the ground-truth images comprises at least two different types of stains.

15. The system of claim 12, wherein the generator machine learning model is trained using a first set of training images comprising microscopic images of sperm cells collected at a first time-period post-coitus and a first set of ground-truth images showing staining that identifies the sperm cells in the first set of training images, and wherein the generator machine learning model is further trained using a second set of training images comprising microscopic images of sperm cells collected at a later time-period post-coitus and a second set of ground-truth images showing staining that identifies the sperm cells in the second set of training images.

16. The system of claim 12, the one or more processors to further execute a post-processing module to determine locations of sperm cells in the generated virtual-stained image of sperm by applying an intensity threshold across locations of the generated virtual-stained image of sperm.

17. The system of claim 16, wherein the post-processing module further determines a quantity of sperm cells in the generated virtual-stained image by determining contours around the locations having an intensity greater than the threshold, wherein each continuous region can be counted as a single sperm cell.

18. The system of claim 12, the one or more processors to further execute a post-processing module to determine a time interval between sperm deposition and sample collection of the sperm cells captured in the microscopic image using a second machine learning model, the second machine learning model takes as input the unstained inference image, one or more generated virtual-stained images of sperm, and the quantity of sperm cells in the one or more generated virtual-stained images, the second machine learning model trained using samples collected at known intervals post-coitus.

19. The system of claim 12, wherein the generator machine learning model generates two or more virtual-stained images of sperm, at least two of the virtual-stained images of sperm comprising different virtual stains.

20. The system of claim 19, the one or more processors to further execute a post-processing module to determine locations of sperm cells in the generated virtual-stained image of sperm by applying an intensity threshold across locations of the generated virtual-stained image of sperm, wherein the locations of sperm cells being where two or more of the stains are above the intensity threshold.
